(19) United States
(12) Patent Application Publication
(10) Pub. No.: US 2016/0097754 A1
(43) Pub. Date: Apr. 7, 2016

(54) MERCURY SENSOR AND METHODS OF USE

(71) Applicant: Duquesne University of The Holy Ghost, Pittsburgh, PA (US)

(72) Inventors: Partha BASU, Pittsburgh, PA (US); Igor PIMKOV, Pittsburgh, PA (US); Kristine DEIBLER, Dillsburg, PA (US)

(73) Assignee: DUQUESNE UNIVERSITY OF THE HOLY GHOST, Pittsburgh, PA (US)

(21) Appl. No.: 14/503,711

(22) Filed: Oct. 1, 2014

(51) Int. Cl. G01N 33/20; G01N 21/76; C07D 495/14

(52) U.S. Cl. CPC G01N 33/20; C07D 495/14; G01N 21/76

(57) ABSTRACT

The invention relates to non-fluorescent or low-fluorescent compounds for contact with mercury ions to produce fluorescent compounds as a detector for mercury. The fluorescence produced by the contact of the non-fluorescent or low-fluorescent compounds with mercury ions has an intensity greater than the intensity produced by the contact of the non-fluorescent or low-fluorescent compounds with other metals. The fluorescent compounds may be used as sensors/detectors for mercury ions in various samples. Methods for detecting and calculating the concentration of mercury ions in samples are also disclosed.

### GOVERNMENTAL INTEREST

This invention was made with government support under grant #GM0061555 awarded by the National Institute of Health (NIH). The government has certain rights in the invention.

### FIELD OF THE TECHNOLOGY

The invention relates to fluorophores that fluoresce with a fluorescence emission when contacted with mercury ions. In particular, the invention includes detectors and sensors, as well as methods for detecting and quantifying mercury ions in a sample.

### BACKGROUND

Fluorescent molecules are of great interest because of their potential uses, for example, but not limited to, in labeling and detection of substrates or molecules in cell-based assays, as components in organic electronic materials in molecular electronics, as pH sensors, and as metal sensors. Molecular fluorescence is a type of photo-luminescence, which is a chemical phenomenon involving the emission of light from a molecule that has been promoted to an excited state by absorption of electromagnetic radiation. Specifically, fluorescence is a luminescence in which the molecular absorption of a photon triggers the emission of a second photon with a longer wavelength (lower energy) than the absorbed photon. The energy difference between the absorbed photon and the emitted photon results from an internal energy conversion of the molecule where the initial excited state (resulting from the energy of the absorbed photon) transitions to a second, lower energy excited state, typically accompanied by dissipation of the energy difference in the form of heat and/or molecular vibration. As the molecule decays from the second excited state to the ground state, a photon of light is emitted from the compound. The energy of the emitted photon is equal to the energy difference between the second excited state and the ground state.

Many fluorescent compounds absorb photons having a wavelength in the ultraviolet portion of the electromagnetic spectrum and emit light having a wavelength in the visible portion of the electromagnetic spectrum. However, the absorption characteristics of a fluorophore are dependent on the molecule's absorbance curve and Stokes shift (difference in wavelength between the absorbed and emitted photon), and some fluorophores may absorb at different portions of the electromagnetic spectrum.

In general, the basic structures of fluorophores may be modified to provide different excitation and emission profiles. For example, fluorescein absorbs electromagnetic radiation having a wavelength of approximately 494 nanometers (“nm”) and emits light having a wavelength at about 525 nm, in the green region of the visible spectrum. Whereas, a related compound rhodamine B absorbs in radiation having a wavelength of about 510 nm and emits light with an emission maximum of about 570 nm, in the yellow-green region of the visible spectrum.

Exposure to mercury can produce toxic effects in high enough doses and can result in mercury poisoning. Toxic effects may include damage to the brain, kidneys and lungs. Mercury poisoning can result in several diseases including acrodynia, Hunter-Russell syndrome and Minamata disease. In particular, children exposed to mercury exposure may develop compromised neurological and/or developmental health. Mercury in varying levels may be found in diverse areas and facilities, such as but not limited to coal production, gold production, nonferrous metal production, cement production, waste disposal, human crematoria, caustic soda production, pig iron and steel production and biomass burning.

Current methods for detecting and quantifying levels of mercury in samples include atomic absorption spectroscopy, inductively coupled plasma mass spectrometry (“ICPMS”), cold vapor atomic absorption spectrometry, and anodic stripping voltammetry. However, these methods have certain disadvantages, including being instrumentally intensive and generally expensive. Thus, there is a need in the art to develop methods and devices for detecting and quantifying levels of mercury in samples that is quick, inexpensive and requires a minimum of instruments or equipment.

### BRIEF SUMMARY

Various embodiments provide for fluorescent compounds that are capable of detecting the presence of mercury in samples. Other embodiments relate to uses of fluorescent compounds as detectors for mercury including the detection of mercury in the presence of other metal ions.

In one embodiment, the invention provides a non-fluorescent or low-fluorescent compound for contact with mercury ions represented by Formula I:

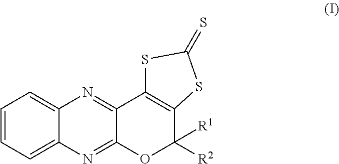

wherein R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl and the substituted phenyl, aryl, or heteroaryl may have from 1 to 5 substituents wherein each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy.

Other embodiments provide for a mercury, e.g., mercury ion, sensor including a matrix material and a fluorophore represented by Formula I, wherein the fluorophore is dissolved in, embedded in, affixed in, absorbed in, or suspended in the matrix material and when in contact with mercury, forms a fluorescent compound represented by Formula II:

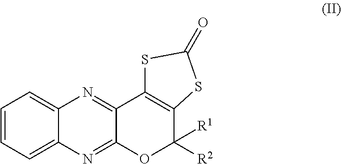

wherein R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl. The substituted phenyl, aryl, or heteroaryl may have from 1 to 5 substituents where each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy.

Further embodiments provide a method for detecting mercury including contacting a mercury sensor including a fluorophore represented by Formula I, with a composition, wherein mercury in the composition is contacted with the fluorophore to produce a fluorescent compound of the Formula II, and measuring a fluorescence emission of the fluoroscent compound. The method may further include exciting the fluorescent compound by irradiating said compound with a light source and/or calculating the concentration of mercury ions in the composition based on the fluorescence emission intensity of the fluoroscent compound.

### BRIEF DESCRIPTION OF THE DRAWINGS

The various embodiments of the invention will be better understood when read with reference to the following figures.

FIG. 1 illustrates a synthetic scheme for the synthesis of an intermediate for the preparation of the fluorophores according to certain embodiments of the invention.

FIG. 2 illustrates synthetic schemes for generating fluorophores possessing structurally distinct formulas according to certain embodiments of the invention.

FIG. 3 is a plot that illustrates fluorescence spectra for a fluorophore according to certain embodiments of the invention as compared to a control sample.

### DETAILED DESCRIPTION

The invention relates to fluorophores that may be synthesized from readily available materials. The structure of the fluorophore is designed with the flexibility to have multiple substitution patterns. Various uses of the fluorophores, including for example, as mercury detectors/sensors, are also disclosed. Particular embodiments include a fluorescent sensor for mercury ions, a fluorophore that can detect mercury ions in the presence of other metal ions in a sample and methods for detecting and quantifying mercury ions in a sample.

Other than the operating examples, or where otherwise indicated, all numbers expressing quantities of ingredients, processing conditions and the like used in the specification and claims are to be understood as being modified in all instances by the term “about”. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, may contain certain errors, such as, for example, equipment and/or operator error, necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of less than or equal to 10.

Any patent, publication, or other disclosure material, in whole or in part, that is said to be incorporated by reference herein is incorporated herein only to the extent that the incorporated material does not conflict with the existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statement, or other disclosure material set forth herein will only be incorporated to the extent that no conflict arises between that incorporated material and the existing disclosure material.

The present invention describes several different features and aspects of the invention with reference to various exemplary non-limiting embodiments. It is understood, however, that the invention embraces numerous alternative embodiments, which may be accomplished by combining any of the different features, aspects, and embodiments described herein in any combination that one of ordinary skill in the art would find useful.

The invention relates to the use of precursor materials, e.g., precursors to fluorophores, having the general structure represented by Formula I.

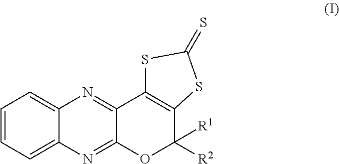

In Formula I, R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl. The substituted phenyl, aryl, or heteroaryl may have from 1 to 5 substituents where each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy. As used herein, the terms “aryl” or “aryl ring” include an aromatic ring (i.e., a single aromatic ring) or ring system (i.e., a polycyclic aromatic ring system) in which all ring atoms are carbon. As used herein, the terms “heteroaryl” or “heteroaryl ring” include an aromatic ring (i.e., a single aromatic ring) or ring system (i.e., a polycyclic aromatic ring system) in which at least one of the ring atoms is a heteroatom, such as nitrogen, oxygen or sulfur heteroatom.

In certain embodiments, R¹ and R² are both methyl (CH₃).

Further, the invention relates to fluorophore precursors contacted with mercury in the presence of acid to form fluorophores which may have the general structure represented by Formula II:

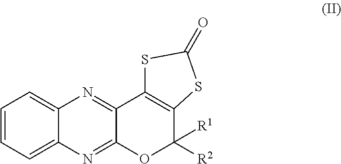

In Formula II, R¹ and R² are as set forth above for Formula I.

In certain embodiments, R¹ and R² are both methyl (CH₃).

Furthermore, in certain embodiments, the precursors materials of the invention may have a general structure as represented by Formulas III, IV and V:

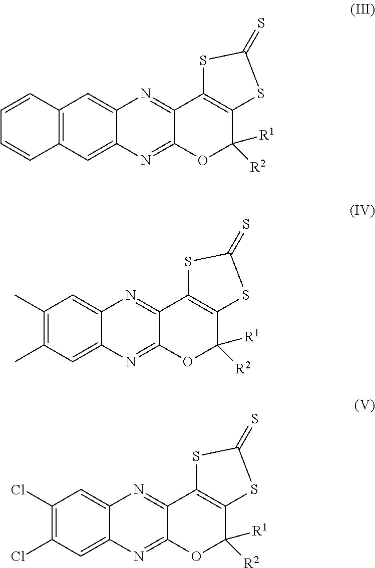

In Formulas III, IV and V, R¹ and R² are as set forth above for Formula I.

In certain embodiments, R¹ and R² are both methyl (CH₃).

Moreover, in certain embodiments, the fluorophores of the invention may have a general structure as represented by Formulas VI, VII and VIII:

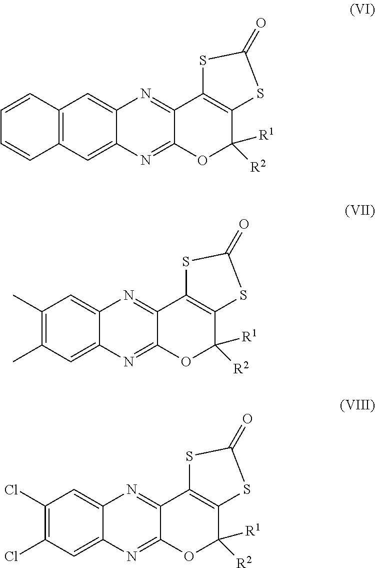

In Formulas VI, VII and VIII, R¹ and R² are as set forth above for Formula I.

In certain embodiments, R¹ and R² are both methyl (CH₃).

As a result of contacting the fluorophore precursors with mercury and acid, fluorophores are formed which exhibit fluorescence. That is, the fluorophores of the invention absorb electromagnetic radiation. Upon absorption of the electromagnetic radiation, the frontier electron (for single electron excitation) of the fluorophores is promoted to an excited electronic state, which then decays to a second excited electronic state concomitant with molecular vibration and/or the release of heat. The fluorophores decay from the second excited state to the ground electronic state with the emission of electromagnetic radiation, wherein the emitted electromagnetic radiation has a wavelength that is longer than the wavelength of the absorbed radiation. For example, certain embodiments of the fluorophores having the structures set forth herein may absorb electromagnetic radiation having a wavelength within the ultraviolet region of the electromagnetic spectrum and fluoresce, that is emit electromagnetic radiation, at a wavelength within the blue light region of the visible spectrum. In certain embodiments, the fluorophores of the invention may fluoresce with an emission maximum at a wavelength within the ultraviolet or visible regions of the electromagnetic spectrum. According to certain embodiments, the fluorophores of the invention may fluoresce with an emission maximum at a wavelength from 200 nm to 850 nm. According to other embodiments, the emission maximum may be at a wavelength from 300 nm to 600 nm. According to other embodiments, the emission maximum may be at a wavelength from 400 nm to 500 nm. As used herein, the term “emission maximum” means the wavelength of the greatest intensity within the fluorescence spectrum of a fluorophore.

Without intending to be limited by any theory or interpretation, it is believed by the inventors that the fluorescent character of the core structure of the fluorophores disclosed herein may depend on and may be manipulated by changing the nature of the conjugated pi system of the fluorophore precursor material, the atoms present in the fluorophore precursor, and/or the substituents attached to the fluorophore precursor. As used herein, the term “fluorescent character” includes such characteristics of the fluorophore, such as, but not limited to, wavelength of light absorbed, the wavelength of the fluorescence emission, the fluorescence emission intensity, and quantum yield. Thus, the fluorescent character of the fluorophores of the invention may be affected by changing one or more of the following: the nature of the pi system of the fluorophore precursor, the atoms in the fluorophore precursor, or the substitution pattern on the fluorophore precursor.

Changes in the conjugated pi system of the fluorophore having Formula I may be affected, for example, by extending the conjugated pi system of the fluorophore, such as by fusing a benzo group (substituted or unsubstituted), an aryl group (substituted or unsubstituted), or a heteroaryl group (substituted or unsubstituted) to the pyrazine ring of the fluorophore (for example, but not limited to, as set forth in Formula II or III). Alternatively, or additionally, the conjugated pi system of the fluorophore may be extended by attaching a conjugation extending substituent to the pyrazine ring or an aromatic or a heteroaromatic ring fused to the pyrazine ring. The aromatic or heteroaromatic ring may be fused directly to the pyrazine ring (that is the rings share two common atoms) or fused indirectly to the pyrazine ring (that is the aromatic or heteroaromatic ring may be fused to an aromatic or heteroaromatic ring that is fused directly or indirectly) to the pyrazine ring. For example, attaching a substituent, such as an electron withdrawing group or an electron donating group, directly to the pyrazine ring or aromatic ring would alter the electronic nature of the conjugated pi system of the fluorophore. As used herein, the term “electron withdrawing group” means a substituent which withdraws electron density from the fluorophore. As used herein, the term “electron donating group” means a substituent that donates electron density into the fluorophore. Altering the conjugated pi system of the fluorophore, such as, by extending the pi system and/or attaching an electron donating group or electron withdrawing group may change the fluorescent character of the fluoropheore, such as, by changing the wavelength of light absorbed and/or emitted or changing the fluorescent quantum yield. As used herein, the term “fluorescence quantum yield” is a measurement of the efficiency of the fluorescence process and is defined by the ratio of the number of photons emitted to the number of photons absorbed by the fluorophore.

In addition, changing the substitution pattern on the fluorophore may also change the fluorescence characteristics of the fluorophore. For example, as set forth herein, attaching a substituent, such as, an electron donating group or an electron withdrawing group may affect the fluorophore and therefore, change the fluorescence characteristics of the fluorophore. Alternatively, or in addition, changing the substitution pattern on the fluorophore may also include changing the nature of the substituent R¹ and/or R². For example, changes in the nature of the substituent at R¹ and/or R² may affect the energy of the excited state of the fluorophore, the wavelength of the radiation absorbed or emitted, and/or the fluorescent quantum yield. In addition, changing the position of one or more substituent on the ring system of the fluorophore (i.e., changing the ring atom that the one or more substituent is bonded to) may change the fluorescence characteristics of the fluorophore.

According to certain embodiments, the fluorophore precursors and fluorophore products may be used as markers and sensors for mercury ions, for example, to determine the presence of mercury ions. For example, when the fluorophore precursor of the Formula I is contacted with mercury ions and acid, significant fluorescence is observed in the fluorophore product of the Formula II. The intensity of the fluorescent emission spectrum of the fluorophore product may be determined. According to specific embodiments, the fluorescent emission spectrum of the fluorophore product may be qualitatively used to determine the presence of a mercury ion in a solution or, alternatively, may be quantitatively used (for example, by the intensity of the emission spectrum) to determine the concentration of the mercury ion in the composition. For example, the intensity of fluorescence can vary according to the concentration of the fluorophore product. Thus, the fluorophore precursors products of the invention can be used as potential markers or sensors for mercury ions.

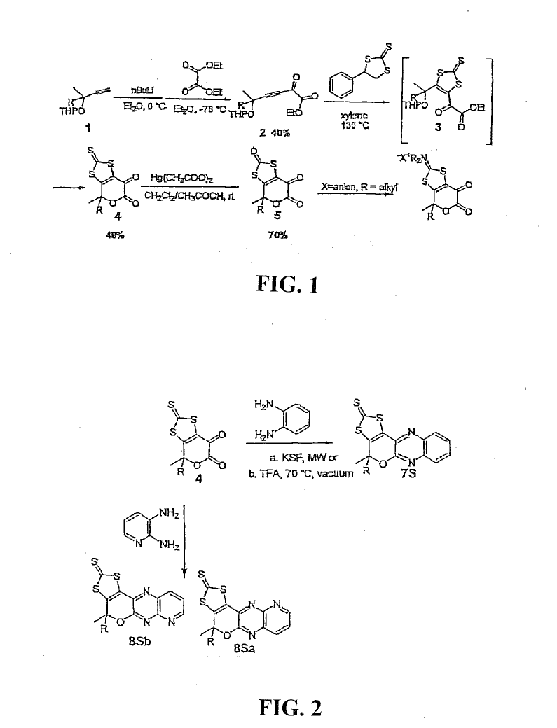

According to various embodiments, the fluorophore precursors of the invention may be readily synthesized using organic chemistry techniques. For example, the synthesis of various embodiments of the fluorophore precursors is described herein. Further, suitable syntheses are disclosed in U.S. Pat. No. 7,888,506 B2, which is incorporated by reference herein. It should be noted that the featured embodiments are intended to be exemplary and are in no way limiting to the scope of the fluorophore precursors and products as described herein. Certain specific examples are discussed in detail in FIGS. 1 and 2.

As illustrated in FIG. 1, the synthetic approach begins with the protection of the substituted propargyl alcohol with a protecting group resulting in alkyne 1. The terminal alkyne in compound 1 is then deprotonated with n-butyl lithium and the reaction of the resulting acetylide with diethyl oxalate at a low temperature yields keto ester 2. The presence of an electron-withdrawing group (i.e., the ketone) activates the alkyne functionality toward the reaction with styrene trithiocarbonate to introduce the protected dithiolene moiety. When the reaction is performed neat, the open intermediate 3 is isolated and then transformed to the pyran-dione 4 upon addition of trifluoro-acetic acid. Conversely, when the reaction is performed in xylene, the pyradione 4 is isolated directly.

Once the diketo-compound 4 is prepared, it may be reacted with a variety of diamines to produce different sets of compounds as desired. In certain embodiments, the condensation reactions of the diketo-compound 4 and diamine resemble those that are known in the art, such as but not limited to, the Isay synthesis of pteridines. Non-limiting examples of synthetic schemes for such reactions are shown in FIG. 2.

In certain embodiments, as shown in FIG. 2, the diketo-compound 4 may be condensed with 2,3-diamino pyridine (e.g., using microwave irradiation) to form the pair of isomeric fluorophores 8Sa and 8Sb, which may be separated. In other embodiments, as shown in FIG. 2, the diketo-compound 4 may be condensed with 1,2-diamino benzene (e.g., using microwave irradiation) to form fluorophore 7S.

The fluorophore precursor compound, such as compounds 7S, 8Sa and 8Sb as shown in FIG. 2, is combined with an acid. The acid is for use in contacting the fluorophore precursor with mercury ions. Suitable acids can be selected from a variety of solvents known in the art. Non-limiting examples of suitable acids and solutions include, but are not limited to, acetic acid, acetic acid-water, hydrochloric acid, hydrochloric acid-water, and mixtures thereof.

In one embodiment, a fluorescing sensor for mercury ions in samples is disclosed. The disclosed compounds, mercury sensors and methods provide cost effective, portable, rapid and reliable methods for detecting and quantifying mercury content in samples that are highly sensitive and selective for mercury over other metal ion contaminants.

Both the fluorophore precursor and the fluorophore product are soluble in aqueous solution making the system suitable for measuring mercury ion concentrations in aqueous samples. In addition, the fluorophore product fluoresces with greater intensity over a wide pH range in aqueous solutions.

When the fluorescing sensor is in the presence of mercury ions, the material fluoresces with a high optical brightness. According to certain embodiments, the fluorophore precursor selectively interacts with mercury ions over other metal ions including other transition metal ions. According to various embodiments, the fluorescence emission intensity of the mercury ion/fluorophore precursor interaction is greater than a fluorescence emission intensity of other metal ion/fluorophore precursor interactions. Other metal ions that may be present in a sample and have a lower fluorescence emission intensity than that produced by the interaction of mercury ions and the fluorophore precursor include but are not limited to lithium ions, sodium ions, potassium ions, calcium ions, magnesium ions, iron ions, cobalt ions, copper ions, zinc ions, manganese ions, tin ions and mixtures thereof. For example, the interaction of mercury ions with the fluorophore precursor in the presence of the other metal ions may have a fluorescence emission intensity at least about 10 times or 20 times greater than the fluorescence emission intensity produced by the interaction of another metal ion with the fluorophore precursor. Since the mercury ion/fluorophore precursor interaction fluoresces with a significantly greater intensity than other metal ion/fluorophore precursor interactions, the fluorescent fluorophore may serve as a selective detector for mercury ions in various samples, such as, samples that may contain other metal ions.

According to certain embodiments, a mercury ion sensor in accordance with the invention includes a matrix material and a fluorophore precursor represented by Formula I, wherein the fluorophore precursor is dissolved in, embedded in, affixed in, absorbed in, or suspended in the matrix material and forms a fluorescent compound represented by the Formula II when interacted with mercury ions in the presence of acid.

The fluorescence emission intensity of the fluorophore product (represented by Formula II) may be measured and compared to a standard calibration plot or values to determine the mercury ion concentration in the sample.

The matrix material may be any material suitable for dissolving, embedding, affixing, absorbing, or suspending the fluorophore that can be used to test a sample composition for mercury ion concentration. For example, the matrix material may be, but is not limited to, a material selected from the group consisting of an aqueous solvent, a gel, a sol-gel material, a solvent, a paper, a polymer, a nanoparticle, a solid state material, and a surface-modified material. One having ordinary skill in the art will recognize that other matrix materials may be used without departing from the intent of the invention.

In certain embodiments of the mercury ion sensor, the sensor may further include a device capable of measuring an intensity of a fluorescence emission spectrum. According to these embodiments, the device may be used to measure the fluorescence emission spectrum of the mercury ion/fluorophore in the matrix material. Examples of devices include, but are not limited to, fluorophoric devices and spectrometers, such as fluorescence spectrometers or fluorometers, laser fluorescence spectrometers, and the like. The fluorescence emission spectrum may be compared to emissions of known standards, for example a calibration plot, to determine the concentration of mercury ions in a sample. For certain embodiments, the determination of the mercury ions may be automated, such as by use of a computer, sampler, or other electronic device. For example, the computer or other electronic device may compare the fluorescence emission spectrum with the spectra of standards and determine the mercury ion concentration in the sample. In other embodiments, the sample and standard spectrum? may be compared by a user of the sensor and a mercury ion concentration of the sample may be determined based on the emission intensity of the mercury ions/fluorophore.

Further embodiments of the invention provide methods for detecting mercury ion concentrations. Accordingly, the methods may include contacting a mercury ion sensor including a fluorophore precursor represented by Formula 1 with a composition, wherein at least a portion (and in certain embodiments all or substantially all) of the mercury ions in the composition interact with the fluorophore to form a fluorophore product represented by Formula II, and measuring a fluorescence emission intensity of the product.

In certain embodiments, the method may further include irradiating the fluorophore with electromagnetic radiation having a wavelength equal to the excitation wavelength or band of the mercury ion/fluorophore product.

The mercury ion sensors and detectors of the invention may be used to sample a wide and diverse variety of compositions. In particular, these sensors and detectors are useful in the energy sector, such as coal burning power plants due to the presence of mercury ions in coal.

While various specific embodiments have been described herein, the disclosure is intended to cover various different combinations of the disclosed embodiments and is not limited to those specific embodiments described herein. Various embodiments of the disclosure will be better understood when read in conjunction with the following non-limiting Example. The procedures set forth in the Example below are not intended to be limiting herein, as those skilled in the art will appreciate that various modifications to the procedures set forth in the Examples, as well as to other procedures not described in the Example, may be useful in practicing the invention as described herein and set forth in the appended claims.

### EXAMPLES

### Example 1

Four samples were prepared. Sample 1 was a control sample, which contained only the fluorophore precursor material as represented by Formula I, wherein R¹ and R² are both methyl (CH₃). Sample 2 included the fluorophore precursor material of Sample 1 in the presence of mercuric acetate (Hg(CH₃COO)₂) wherein the ratio was 1:0.5, respectively, to produce the fluorophore product material as represented by Formula II, wherein R¹ and R² are both methyl (CH₃). Sample 3 was the same as Sample 2 with the exception that the ratio of fluorophore precursor material to mercuric acetate was 1:1. Sample 4 contained only the fluorophore product material as represented by Formula II, wherein R¹ and R² are both methyl (CH₃). Visual observation of the results showed an absence of fluorescence for Sample 1 and a presence of fluorescence for Samples 2, 3 and 4. The fluorescence of Sample 3 was greater than Sample 2 and was almost equivalent to the fluorescence of Sample 4.

### Example 2

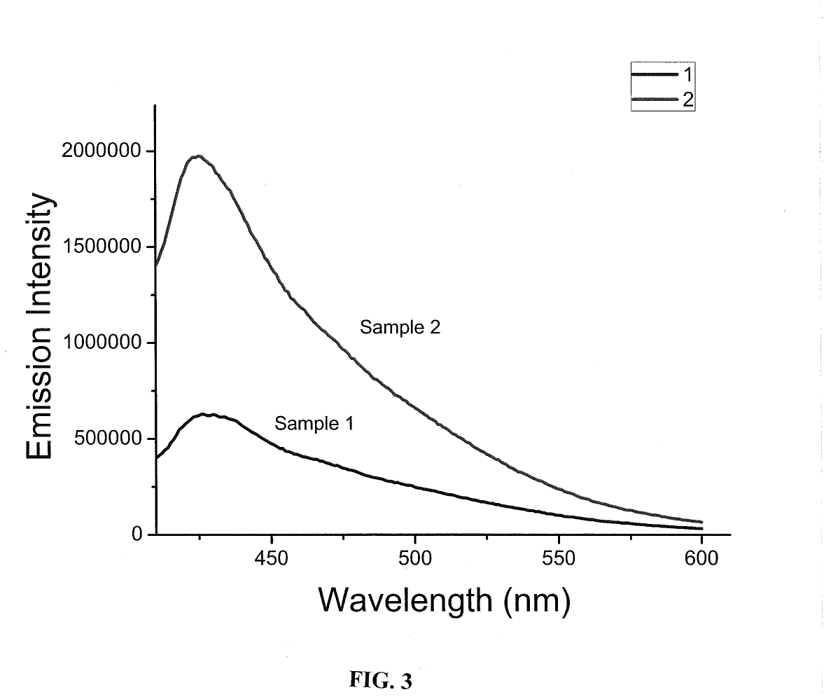

Two samples were prepared. Sample 1 was a control sample which contained 0.5 mL of 1.038×10⁻⁴ M solution of the fluorophore precursor material as represented by Formula I, wherein R¹ and R² are both CH₃, in CH₃COOH and 0.5 ml of CH₃COOH. Sample 2 contained 0.5 mL of 1.038×10⁻⁴M solution of the fluorophore precursor material as represented by Formula I, wherein R¹ and R² are both CH₃, in CH₃COOH and 0.5 ml of 0.94×10⁻⁴M solution of Hg(CH₃COO)₂ in CH₃COOH, to produce the fluorophore product material as represented by Formula II, wherein R¹ and R² are both methyl (CH₃). Both samples were incubated in an oil bath for approximately 15 minutes at a temperature of 80° C. The results are shown in FIG. 3.

We claim:

1. A non-fluorescent or low-fluorescent compound for contact with mercury ions, represented by a structure of Formula I: 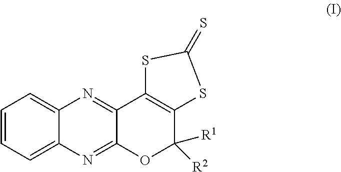
wherein R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl and the substituted phenyl, aryl, or heteroaryl may have from 1 to 5 substituents wherein each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy.

2. The compound of claim 1, wherein R¹ and R² are both CH₃.

3. The compound of claim 1, further comprising a matrix material, wherein said compound is dissolved in, embedded in, affixed in, absorbed in, or suspended in the matrix material and when in contact with mercury, forms a fluorescent compound represented by a structure of Formula II: 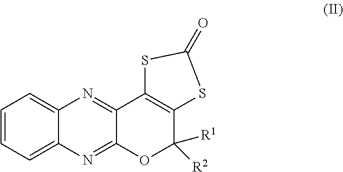
wherein R¹ and R² are as set forth above for Formula I.

4. A fluorescent compound as a detector for mercury, represented by a structure of Formula II: 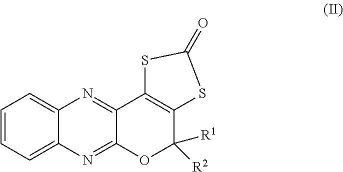
wherein R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl and the substituted phenyl, aryl, or heteroaryl may have from 1 to 5 substituents wherein each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy.

5. The compound of claim 4, wherein R¹ and R² are both CH₃.

6. The compound of claim 4, wherein said compound is effective to detect the presence of mercury in samples.

7. The compound of claim 6, wherein the samples include a presence of other metal ions.

8. The compound of claim 1, wherein said compound is represented by a structure of Formula III: 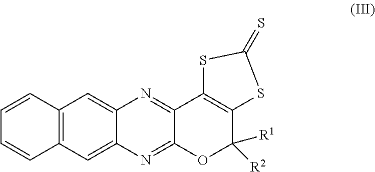
wherein R¹ and R² are as set forth above for Formula I.

9. The compound of claim 1, wherein said compound is represented by a structure of Formula IV: 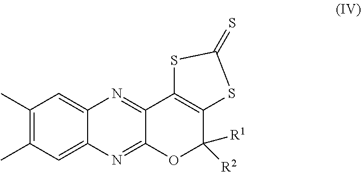
wherein R¹ and R² are as set forth above for Formula I.

10. The compound of claim 1, wherein said compound is represented by a structure of Formula V: 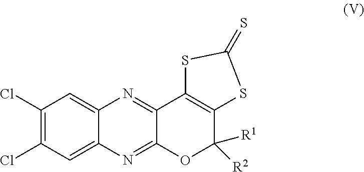
wherein R¹ and R² are as set forth above for Formula I.

11. The compound of claim 4, wherein said compound is represented by a structure of Formula VI: 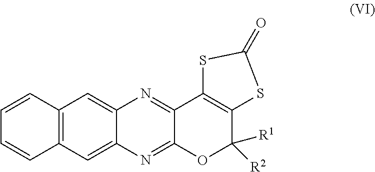
wherein R¹ and R² are as set forth above for Formula II.

12. The compound of claim 4, wherein said compound is represented by a structure of Formula VII: 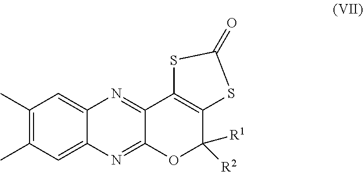
wherein R¹ and R² are as set forth above for Formula II.

13. The compound of claim 4, wherein said compound is represented by a structure of Formula VII: 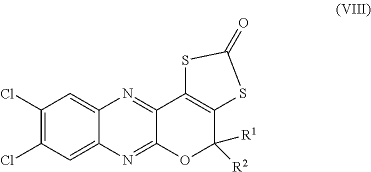
wherein R¹ and R² are as set forth above for Formula II.

14. A method for detecting mercury in a composition, comprising:
providing a mercury sensor, comprising a non-fluorescent or low-fluorescent compound represented by a structure of Formula I: 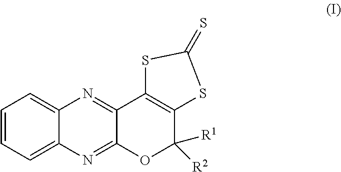
contacting the mercury sensor of the Formula I with the composition;
when mercury in the composition is in contact with the compound of Formula I, producing a fluorescent compound represented by a structure of Formula II: 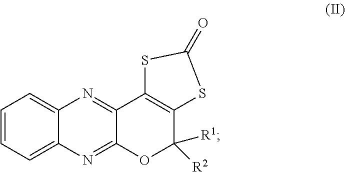
and
measuring a fluorescence emission intensity of the fluorescent compound,
wherein R¹ and R² may each independently be hydrogen, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, phenyl, substituted phenyl, aryl, substituted aryl, heteroaryl, or substituted heteroaryl and the substituted phenyl, aryl, or heteroaryl may have from 1 to 5 substituents wherein each substituent may be one or more of fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy.

15. The method of claim 14, wherein R¹ and R² are both CH₃.

16. The method of claim 14, further comprising exciting the fluorescent compound by irradiating said compound with a light source.

17. The method of claim 14, further comprising calculating the concentration of mercury ions in the composition based on the fluorescence emission intensity of the fluorescent compound.

18. The method of claim 14, wherein the non-fluorescent or low-fluorescent compound is represented by a structure selected from the group consisting of Formulas III, IV and IV: 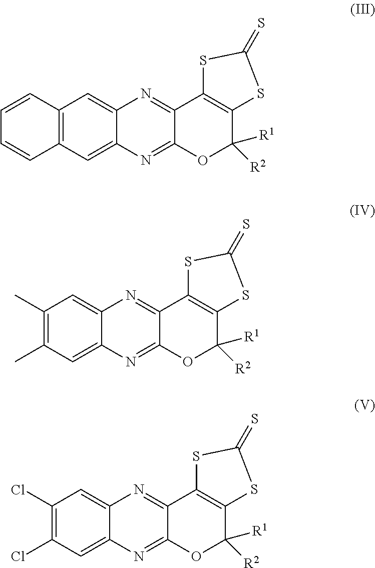
wherein R¹ and R² are as set forth above for Formula I.

19. The method of claim 14, wherein the fluorescent compound is represented by a structure selected from the group consisting of Formulas VI, VII and VIII: 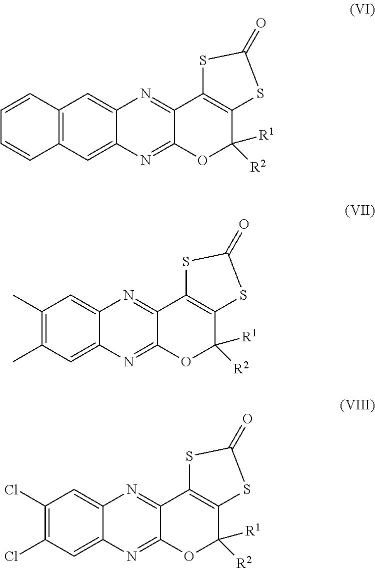
wherein R¹ and R² are as set forth above for Formula II.
